(19) United States
(12) Patent Application Publication
(10) Pub. No.: US 2006/0264982 A1
(43) Pub. Date: Nov. 23, 2006

(54) Gastric restrictor assembly and method of use

(76) Inventors: Frank J. Viola, Sandy Hook, CT (US); Brian J. Creston, Milford, CT (US)

Correspondence Address: UNITED STATES SURGICAL,;A DIVISION OF TYCO HEALTHCARE GROUP LP, 195 MCDERMOTT ROAD, NORTH HAVEN, CT, 06473, US

(21) Appl. No.: 11/133,605

(22) Filed: May 20, 2005

(51) Int. Cl. A61B 17/08

(52) U.S. Cl. 606/153

(57) ABSTRACT

A gastric restrictor assembly for positioning about a portion of the digestive tract, e.g., stomach, esophagus, etc., of a patient to treat obesity is disclosed. The gastric restrictor assembly includes an inner expandable member and an outer collar. The inner expandable member is dimensioned to be transesophagealy positioned within the digestive tract, e.g., stomach. The outer collar is dimensioned to be positioned about the digestive tract, e.g., the stomach, and the inner expandable member to clamp the stomach therebetween. The inner expandable member defines an opening therethrough. The diameter of the opening is selectively adjustable via a fill tube to selectively control the diameter of the central opening.

### BACKGROUND

1. Technical Field

The present disclosure relates to a surgical implant for treating obesity in a patient. More particularly, the present disclosure relates to a surgical implant for constricting the stomach of a patient to treat obesity in a patient.

2. Background of Related Art

A variety of different approaches are known for the treatment of obesity in a patient. These approaches can be of the nonsurgical variety, e.g., dieting, or of the surgical variety, e.g., gastric bypass, small bowel bypass etc. Where non-invasive non-surgical procedures such as dieting rely on the will power of a patient and may not be effective, invasive surgical procedures such as bypass surgery can be risky and have undesirable side effects. As such, less invasive surgical devices for constricting or reducing the capacity of the stomach have been developed. These devices include gastric bands which are positioned about the stomach to constrict the stomach and devices such as inflatable balloons for reducing the reservoir capacity of the stomach. Each of these types of devices produce a sense of satiety in a patient to reduce the patient's desire to ingest food.

Although the above-identified devices have had some success, improvements to these devices would be desirable.

### SUMMARY

In accordance with the present disclosure, a gastric restrictor assembly is provided which includes an inner expandable member and an outer collar. The inner expandable member is dimensioned to be received within a portion of the digestive tract, e.g., the stomach, and includes a valved port dimensioned to mateably receive one end of a fill tube. The fill tube is associated with a fluid source and is operable to inflate or deflate the inner expandable member. In one embodiment, the inner expandable is annular and defines a central opening. The diameter of the central opening is adjustable by operating the fill tube to inflate or deflate the inner expandable member.

In one embodiment, the outer collar is formed of a first half-section and a second half-section which can be fastened together to define the collar. The collar is dimensioned to be positioned about an external wall of the stomach adjacent the inner expandable member to clamp a wall of the stomach therebetween.

In one embodiment, an inner wall of the collar defines a concavity dimensioned to receive the inner expandable member to clamp tissue therebetween and prevent axial movement of the inner expandable member in relation to the outer collar and the stomach.

In one embodiment, the inner wall of the outer collar and/or the outer wall of the inner expandable member is textured to minimize slippage of the gastric restrictor assembly in relation to the stomach. In another embodiment, magnets may be provided on the inner expandable member and the outer collar to facilitate positioning of inner expandable member in relation to outer collar during installation of the gastric restrictor assembly within the body of a patient.

### BRIEF DESCRIPTION OF THE DRAWINGS

Various embodiments of the presently disclosed gastric restrictor assembly are disclosed herein with reference to the drawings, wherein:

FIG. 1 is a top side perspective exploded view of one embodiment of the presently disclosed gastric restrictor assembly;

FIG. 2 is a side cross-sectional view of the gastric restrictor assembly positioned about a portion of the stomach prior to attachment of a control member to the expandable member with the expandable member in a first state of expansion;

FIG. 3 is a side cross-sectional view of the gastric restrictor assembly positioned about a portion of the stomach with the control member attached to the expandable member and the expandable member in a second state of expansion; and

FIG. 4 is a top, side perspective exploded view of another embodiment of the presently disclosed gastric restrictor assembly.

### DETAILED DESCRIPTION OF EMBODIMENTS

Embodiments of the presently disclosed gastric restrictor assembly and its method of use will now be described in detail with reference to the drawings in which like numerals designate identical or corresponding elements in each of the several views:

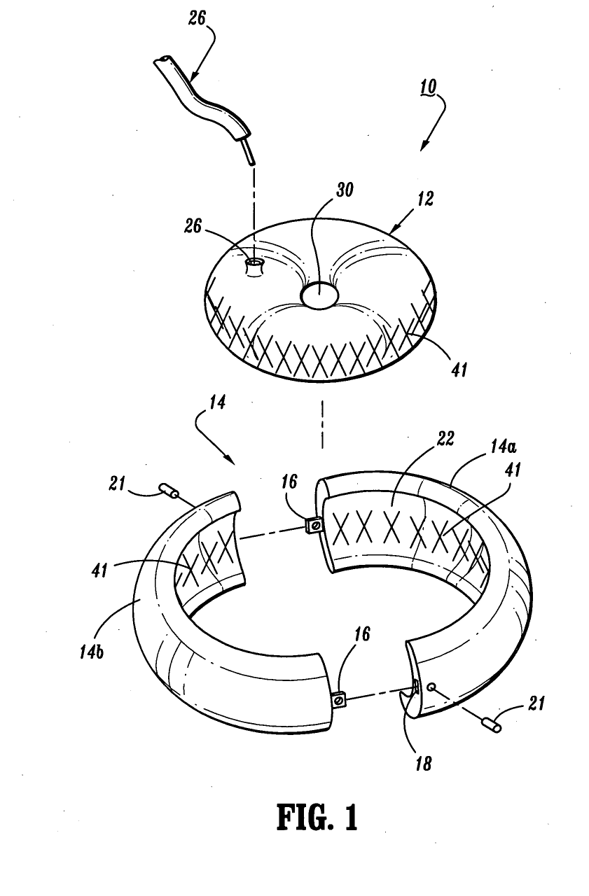

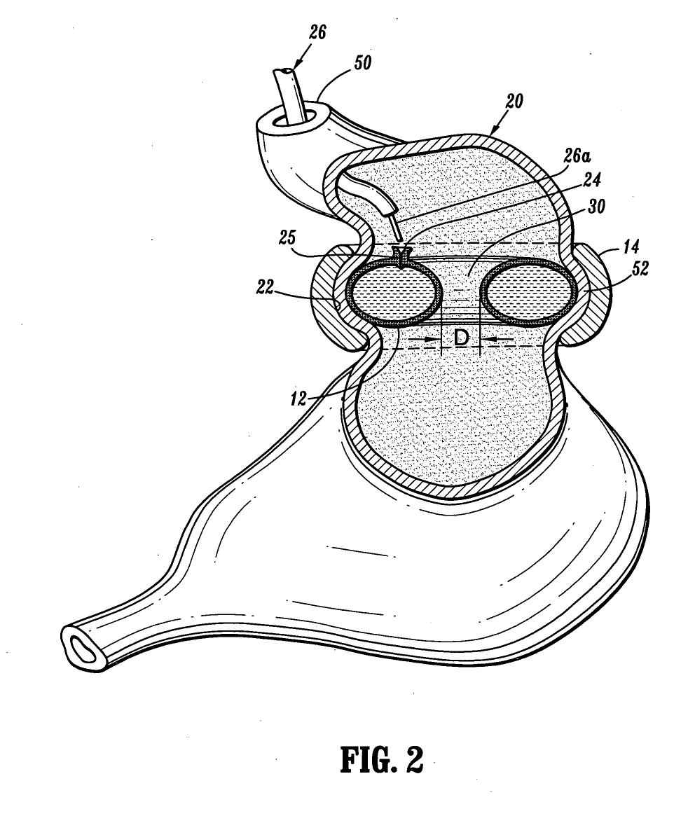

FIG. 1 illustrates one embodiment of the presently disclosed gastric restrictor assembly shown generally as 10. Gastric restrictor assembly 10 includes an inner expandable member 12 and an outer collar 14. Outer collar 14 can be formed from a plurality of sections, e.g., a first half-section 14a and a second half-section 14b, which are connectable together about the stomach 20 (FIG. 2). In one embodiment, each of half-sections 14a and 14b includes a tab 16 and a recess 18 which is dimensioned to receive a respective tab 16 formed on the other half-section to secure half-section 14a to half-section 14b about stomach 20 (FIG. 2). A pin 21 is provided to secure each tab 16 within each recess 18. The use of other known fastening techniques including adhesives, pins, screws, interlocking structure, etc. is also contemplated for fastening half-section 14a and half-section 14b.

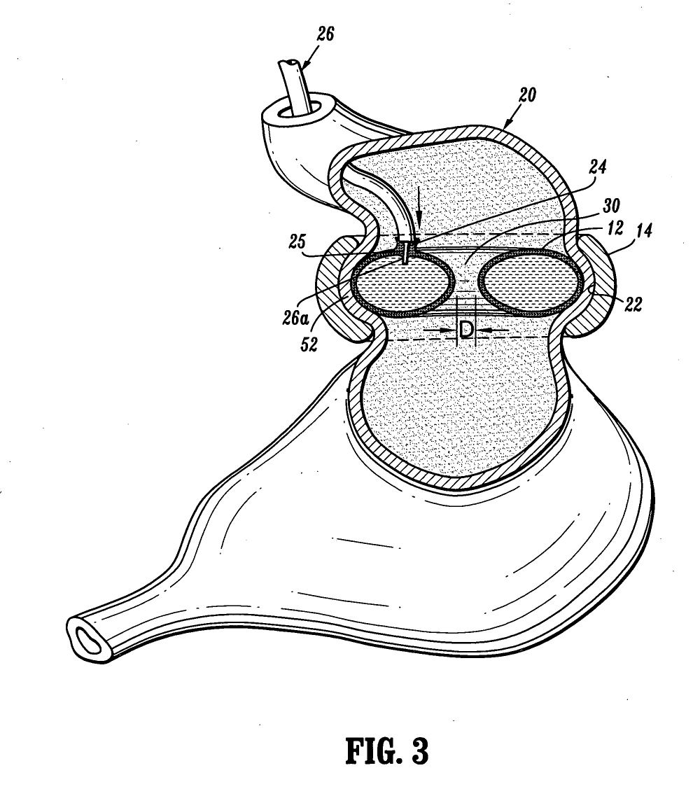

In one embodiment, collar 14 is formed from a substantially rigid material, e.g., a polyetheretherketone (PEEK) material and has an inner surface which defines an annular recess or concavity 22 configured to receive expandable member 12 as shown in FIGS. 2 and 3. Alternately, other materials of construction are envisioned including semi-rigid and resilient materials, e.g., rubber, polycarbonate, etc. Concavity 22 prevents expandable member 12 from moving axially in relation to outer collar 14 and provides a surface for receiving and clamping a portion of stomach 20.

Referring also to FIGS. 2 and 3, inner expandable member 12 is formed from an expandable material which is capable of withstanding stomach acids, e.g., polyethylene, polytetrafluoroethylene (Teflon®), etc. A port 24 is provided in a top surface of expandable member 12. Port 24 includes a valve 25 which is configured to receive one end 26a (FIG. 2) of a control member or fill tube 26 to control the amount of fluid located within expandable member 12. By controlling the amount of fluid within expandable member 12, the diameter “D” of opening 30 (FIGS. 2 and 3) defined by expandable member 12 can be regulated. For example, by adding fluid to expandable member 12 as shown in FIG. 2, the diameter D of opening 30 can be reduced as shown in FIG. 3. Alternately, fluid can be removed from expandable member 12 to increase the diameter of opening 30. In one embodiment, control member 26 has a second end (not shown) which communicates with a pump and a source of fluid (not shown) such as a liquid, e.g., saline, mineral oil, water, etc., or a gas, e.g., air, nitrogen, C0₂ etc. The pump is reversible and is capable of supplying liquid to or withdrawing liquid from control member 26.

Although the wall thickness of expandable member 12 is illustrated in FIG. 2 as being uniform, it is contemplated that the wall thickness near or defining opening 30 may be reduced as compared to the remaining portion of expandable member 12. By reducing the wall thickness of member 12 adjacent opening 30, expandable member 12 can be constructed to expand primarily inwardly during inflation of member 12.

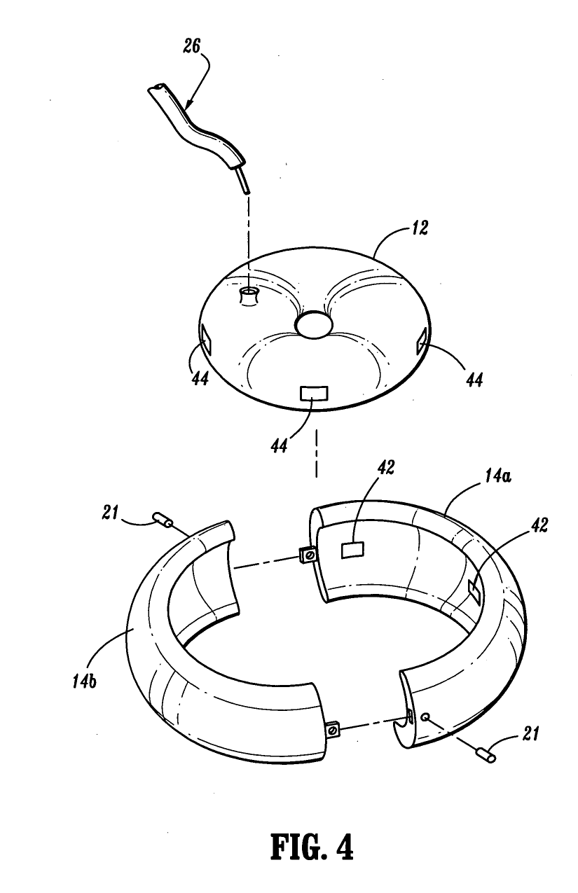

In one embodiment, the outer surface of expandable member 12 and/or the inner surface of collar 14 can be textured, e.g., knurled 41, to minimize slippage of gastric restrictor assembly 10 about the stomach. In another embodiment shown in FIG. 4, the inner surface of collar 14 and the outer surface of expandable member 12 each include one or more magnets 42 and 44, respectively, which are attracted to the other. The magnets 42 and 44 function to assist placement of expandable member 12 within collar 14 during installation of the gastric restrictor assembly 10. Alternately, magnets 42 and 44 may be annular and extend partly or entirely about expandable member 12 and/or collar 14.

Referring to FIGS. 2 and 3, during installation of gastric restrictor assembly 10 on and within stomach 20, collar half-sections 14a and 14b are laparoscopically (or in the alternative during an open procedure) positioned about a desired portion of stomach 20 and secured together using, for example pins 21 (FIG. 1). Next, expandable member 12 is inserted transesophegealy, in a substantially deflated condition, into stomach 20. Thereafter, distal end 26a of fill tube 26 is inserted through a patient's mouth (not shown) and esophagus 50 and into valve 25 of part 24 to inject fluid into expandable member 12. The position of expandable member 12 is controlled such expandable member 12 expands into concavity 22 of collar 14 to clamp a wall 52 of stomach 20 between expandable member 12 and collar 14. Fill tube 26 facilitates selective adjustment of the diameter “D” of opening 30 of expandable member 12 in a minimally invasive manner. By clamping a portion of stomach 20 between collar 14 and expandable member 12, the likelihood of slippage of restrictor assembly 10 about stomach 20 is minimized.

It will be understood that various modifications may be made to the embodiments disclosed herein. For example, although not discussed herein, the gastric restrictor assembly may also be positioned about the lower portion of the esophagus adjacent the stomach or, alternately, about any portion of the digestive tract where restriction may be warranted or desired. Therefore, the above description should not be construed as limiting, but merely as exemplifications of embodiments. Those skilled in the art will envision other modifications within the scope and spirit of the claims appended hereto.

What is claimed is:

1. A gastric restrictor assembly comprising:
an inner expandable member defining an opening therethrough and being dimensioned to be positioned within the stomach; and
an outer collar configured and dimensioned to be positioned about an outer wall of the stomach;
wherein the inner expandable member is expandable towards the outer collar to clamp the stomach between the inner expandable member and the outer collar.

2. A gastric restrictor assembly according to claim 1, wherein the outer collar includes a plurality of sections which are fastened together to form an annular member.

3. A gastric restrictor assembly according to claim 1, wherein the outer collar is substantially rigid.

4. A gastric restrictor assembly according to claim 1, wherein the outer collar is formed from a flexible material.

5. A gastric restrictor assembly according to claim 1, wherein the outer collar includes an inner surface defining a concavity configured to receive the inner expandable member.

6. A gastric restrictor assembly according to claim 1, wherein an inner surface of the outer collar and/or an outer surface of the inner expandable member is textured to provide a slip resistant surface.

7. A gastric restrictor assembly according to claim 1, wherein an inner surface of the outer collar and an outer surface of the inner expandable member each include at least one magnetic member to assist positioning of the inner expandable member in relation to the outer collar.

8. A gastric restrictor assembly according to claim 1, wherein the inner expandable member includes a port adapted to mate with a fill tube to facilitate inflation and deflation of the inner expandable member.

9. A gastric restrictor assembly according to claim 1, wherein the inner expandable member is constructed from a material selected from the group consisting of polyethylene, polyproplene and polytetrafluoroethylene.

10. A gastric restrictor assembly according to claim 1, wherein the inner expandable member is annular and the opening is a central opening.

11. A gastric restrictor assembly according to claim 10, wherein the inner expandable member is inflatable and deflatable to selectively change the diameter of the central opening.

12. A method for treating obesity comprising the following steps:
a) positioning an expandable member defining an opening within a portion of the digestive tract;
b) positioning a collar about the portion of the digestive tract;
c) expanding the expandable member towards the collar to clamp the portion of the digestive tract between the expandable member and the collar.

13. A method according to claim 12, wherein the portion of the digestive tract is a stomach.

14. A method according to claim 13, wherein the collar includes a plurality of sections which are fastened together to form an annular member.

15. A method according to claim 13, wherein the collar is substantially rigid.

16. A method according to claim 13, wherein the collar includes an inner surface defining a concavity configured to receive the expandable member.

17. A method according to claim 13, wherein the collar is formed from a flexible material.

18. A method according to claim 13, wherein an inner surface of the collar and/or an outer surface of the expandable member are textured to provide a slip resistant surface.

19. A method according to claim 13, wherein an inner surface of the collar and an outer surface of the expandable member each include at least one magnetic member to assist in positioning of the expandable member in relation to the collar.

20. A method according to claim 13, further including the step of inflating or deflating the expandable member to change a diameter of the opening.
